(19) United States
(12) Patent Application Publication
(10) Pub. No.: US 2011/0318760 A1
(43) Pub. Date: Dec. 29, 2011

(54) METHOD FOR THE IN VITRO DIAGNOSIS OF AN AUTOIMMUNE RESPONSE ASSOCIATED WITH THE EXISTENCE OF AN ANTINEUTROPHIL CYTOPLASMIC ANTIBODIES-ASSOCIATED VASCULITIS BY DETECTION OF ANTIBODIES DIRECTED AGAINST THE PENTRAXIN 3 ANTIGEN

(75) Inventors: Pascale Jeannin, Bouchemaine (FR); Yves Delneste, Bouchemaine (FR); Alberto Mantovani, Milan (IT)

(73) Assignee: Universite d'ANGERS, Angers (FR)

(21) Appl. No.: 13/161,158

(22) Filed: Jun. 15, 2011

Related U.S. Application Data
(63) Continuation-in-part of application No. 12/223,004, filed on Jul. 18, 2008, now abandoned.

(30) Foreign Application Priority Data
Jan. 20, 2006 (FR) 06/00516

(51) Int. Cl. G01N 33/566

(52) U.S. Cl. 435/7.92; 436/501

(57) ABSTRACT

A method for the in vitro diagnosis of an autoimmune immune response in an individual by detection, in a biological fluid from said individual, of antibodies directed against the pentraxin 3 (PTX3) antigen, characterized in that the presence of antibodies directed against the PTX3 antigen (anti-PTX3 antibodies) are determined in a biological fluid from the individual, and kits for implementing the method.

This application is a Continuation-in-part of U.S. patent application Ser. No. 12/223,004, filed on 18 Jul. 2008, which claims priority to International Patent Application PCT/EP2007/050562, filed on 19 Jan. 2007. The co-pending parent application is hereby incorporated by reference herein in its entirety and is made a part hereof, including but not limited to those portions which specifically appear hereinafter.

The invention relates to a method for in vitro diagnosis of an autoimmune response in a subject by detection in the biological fluid of said subject of antibodies directed against the pentraxin 3 (PTX3) antigen characterised in that the presence of antibodies directed against the PTX3 antigen (anti-PTX3 antibodies) is determined. More particularly, the method of the invention relates to the in vitro diagnosis of an autoimmune response associated with the existence of an antineutrophil cytoplasmic antibodies(ANCA)-associated vasculitis in a subject presenting clinical symptoms of said vasculitis. It also relates to kits for implementing this method.

### I. STATE OF THE ART

### 1. Lymphocytes in the Immune Response

a. Lymphocytes and the Anti-Self Physiological Immune Response

The role of cells of the immune system, particularly lymphocytes (B and T), is the destruction of foreign agents such as microorganisms in the body. Lymphocytes express receptors which recognise microbial agents. Thus for example, T lymphocytes express the T receptor and B lymphocytes express immunoglobulins, also called antibodies. When activated, lymphocytes specific for microorganism constituents release mediators which destroy microorganisms and/or cells infected by microorganisms.

Broadly speaking, these mediators are immunoglobulins (or antibodies) in the case of B lymphocytes. These antibodies bind to microorganisms and/or to infected cells and stimulate their elimination by other immune cells such as macrophages, polynuclear or natural killer cells, also called NK cells. The mediators that facilitate the elimination of microorganisms produced by T lymphocytes are cytokines and/or toxic mediators.

Thus, recognition of a microorganism (considered to be non-self) by specific lymphocytes generally leads to its destruction by cells of the immune system.

b. Autoimmune Processes and Autoimmune Diseases

In addition to B and T lymphocytes which carry receptors that recognise non-self constituents such as microorganisms, all subjects also carry lymphocytes which recognise self-constituents. Under normal physiological conditions, these lymphocytes are not activated. They are maintained by means of various mechanisms in a non-reactive state to the self, called tolerance or anergy.

Under certain ill-defined conditions, for example viral infection or genetic predisposition, these lymphocytes may be activated. This then results in the development of an abnormal and damaging immune response leading to tissue destructions. The immune system, and in particular lymphocytes, act as if cells and/or certain molecules of the subject were foreign and mobilize their whole arsenal to destroy these cells. Consequently, activated lymphocytes specific to the self then react by releasing toxic antibodies and/or mediators.

This lymphocyte reaction against self-constituents is what characterises the autoimmune processes. If this reaction results in the development of a pathology, it is called an autoimmune disease. Nevertheless, an autoimmune type disorder can also be found associated with many other pathologies, such as chronic inflammation or even pathologies associated with considerable cell lysis.

### 2. Autoantibodies

a. Definitions

In the case of an autoimmune type immunological disorder, B lymphocytes specific of the self are activated and produce immunoglobulins that are specific to these constituents. These antibodies are called autoantibodies. Self molecules and/or structures recognised by antibodies are called autoantigens. Self-constituents are expressed by tissues, cells or produced by cells in all subjects. Some of these molecules are structural proteins while others play a well-established role, for example molecules involved in coagulation and/or in destruction/elimination of bacterial constituents.

b. Target Antigens of Autoantibodies.

Currently, a hundred or so of molecules have been identified as being autoantibody targets. Examples of these include:
anti-phospholipid antibodies directed against two plasma proteins bound to anionic phospholipids: β2-glycoprotein I and prothrombin,
soluble anti-nuclear antibodies directed against antigens Sm, Sc170, SSA, SSB, and Jol.,
anti-polynuclear neutrophil antibodies directed against proteins such as myeloperoxidase, proteinase 3 and, less frequently, against the BIP molecule (bactericidal increasing protein), azurocidin, elastase and cathepsin G.
   c. Physiopathological Role of Autoantibodies.

In addition to a central role in the diagnosis of immune disorders, some autoantibodies play an active role in the pathogenicity of the disease. For example, circulating immune complexes deposits (autoantibodies/autoantigen complexes) become deposited in kidneys and cause glomerulonephritis (acute inflammation of kidney). Anti-nucleosome antibodies produced in the course of disseminated lupus erythematosis (DLE) are also directly involved in the production of lesions in kidneys. In some special cases, the presence of autoantibodies changes the function of the autoantigen and has physiopathological consequences.

Nonetheless, at present, while a link between the presence and level of autoantibodies and the existence of organ disorders is suspected, the physiopathological role is not known for the majority of autoantibodies of known specificity.

d. Immunological Diagnosis Allowing Investigation of Autoantibodies.

At present, the most commonly used techniques in biology and immunology laboratories to detect an autoimmune process consist in investigating the presence of autoantibodies in patient sera.

The presence of autoantibodies capable of binding to various tissues, whether human or not, or to cells or proteins is analysed. Non-human tissues are sometimes used when it has been previously demonstrated that the autoantibody target is the same across species.

Techniques used to investigate autoantibodies when the autoantigen source used is tissues or cells are immuno-histochemistry and immuno-cytochemistry techniques respectively: tissue sections or cells are contacted with different dilutions of the serum being tested. After incubation then washing, immunoglobulins which recognise tissues or cells are detected using a human anti-immunoglobulin antibody coupled to a molecule which allows detection such as a fluorochrome or an enzyme-substrate complex.

When the molecules recognised by the autoantibodies are known, other techniques are used: ELISA, Immuno-Dot and/or immunoprints. In the first two cases, the molecule (autoantigen) is adsorbed on a polystyrene plate or a membrane respectively. The ELISA plate or the membrane is incubated with serum and the antibodies present in the serum which bind to the target are detected by means of a human anti-immunoglobulin antibody coupled to a molecule which allows detection such as a fluorochrome or an enzyme-substrate complex. In the case of immunoprints, a total protein extract of a cell or purified molecule migrates in polyacrylamide gel. The molecules thus separated according to their molecular weights are then transferred onto a membrane which undergoes the same process as described above.

e. Diagnostic and Prognostic Benefits of Autoantibody Detection.

The benefits of autoantibody detection reside in the indication of dysimmunity, or in other words of an abnormal immune response allowed by dysimmunity. Identification of autoantibodies is of no diagnostic benefit unless it is coupled to clinical information. In fact, the production of autoantibodies can precede any clinical symptoms. The presence of autoantibodies directed against certain autoantigens is mainly associated with certain so-called immune diseases such as anti-nuclear autoantibodies and DLE. Biological results are generally essential to complement the clinical picture in order to facilitate the therapeutic approach to be used, especially when this picture is atypical, which is often the case when the disease is detected at an early stage. Current treatments such as immunosuppressants and corticosteroids are effective but aggressive. The presence of autoantibodies, their specificity and level is therefore generally essential to assist the clinician in evaluating the risk/benefit ratio of treatments.

f. Current Benefit of Research of New Autoantibody Targets.

At present, research of new autoantibody targets appears to be a choice niche in the field of biotechnology. Autoimmune diseases are the third highest cause of morbidity in industrialised countries. Population ageing contributes to this increase. Research of new targets allows rapid and early diagnosis of an autoimmune response as well as completing the panel of autoantigen targets used in laboratories. There is currently a real interest in developing further diagnostic tests in addition to existing tests to benefit from earlier and more appropriate information in order to follow the course of the disease. In fact, early treatment makes it possible to avoid complications and to limit therapeutic costs of such chronic diseases. It should be noted that rapid diagnostic techniques also seem to be essential when the vital prognosis is at risk, for example in the case of kidney or lung disorders.

This is precisely the object of the present invention.

### II. THE INVENTION

Indeed, the inventors have succeeded in finding a means to respond to this prospect by showing, for the first time, the presence of autoantibodies directed against the pentraxin 3 molecule (PTX3), also called TNF-inducible gene (TSG-14) in the biological fluids of subjects with an autoimmune response, and more particularly of subjects presenting clinical symptoms of an antineutrophil cytoplasmic antibodies(ANCA)-associated vasculitis (AAV).

As a result, the present invention relates to a method for in vitro diagnosis of an autoimmune response in a subject by detection in a biological fluid of said subject of antibodies directed against the pentraxin 3 antigen (PTX3). The present invention more particularly relates to a method for in vitro diagnosis of an autoimmune response associated with the existence of an antineutrophil cytoplasmic antibodies-associated vasculitis in a subject presenting clinical symptoms of said vasculitis.

### I. Pentraxin 3, a Mediator of Innate Immunity.

a. Innate Immune Receptors.

Innate immunity cells are involved in the rapid recognition of microbes. Their activation controls the propagation of microbes, in particular via the production of microbiocidal mediators as well as the development of a specific immune response. In order to avoid recognition by the innate immunity cells they encounter, microbes produce many mutations in order to enhance the heterogeneity of their constituents. In order to counteract this recognition-avoidance strategy, innate immunity cells have selected receptors capable of recognising structures that are highly preserved in microorganisms and necessary for the physiology of microorganisms. These preserved structures such as lipopolysaccharides, double stranded RNA and CpG sequences are grouped together under the term “pathogen-associated molecular pattern” or PAMPs. By definition, PAMPs are distinct from the self, shared by large groups of pathogens and are essential to their survival. They constitute true molecular signatures of microbes and their recognition triggers the antimicrobial immune response.

Receptors involved in the recognition of PAMPs are called innate immune receptors or Pattern-Recognition Receptors (PRRs). PRRs recognise a broad spectrum of microbial constituents such as sugars, proteins, lipids and nucleic acids (Medzhitov and Janeway, 2000; Janeway and Medzhitov, 2002). PRRs are expressed by innate immune cells either at the intracellular or membrane level or in the extracellular environment.

PRRs are distinguished depending on their biological function:
recognition PRRs involved in the detection and/or internalisation of microorganisms by innate immune cells, and
signalling PRRs involved in the activation of immune cells by microorganisms.

Membrane recognition PRRs belong, amongst others, to the family of purging receptors, mannose receptors, C-type lectins and integrins. Soluble recognition PRRs, also called opsonins, belong to the collectins (van de Wetering et al, 2004), ficollins (Matsushita & Fujita, 2002) and pentraxins (Garlanda et al, 2005) family. The role of soluble recognition PRRs is to recognise, bind to and then favour the elimination of microbes by phagocytic cells.

The pentraxin (PTX) superfamily includes molecules that have been highly conserved in the course of evolution characterised by the presence of a “pentraxin” domain in the C-terminal region. It includes many members such as:
C-reactive protein (CRP, also called PTX1) and serum amyloid P (also called PTX2). CRP and SAP are acute phase proteins produced by liver in response to proinflammatory stimuli such as IL-6. CRP and SAP bind to a wide variety of molecules including self (complement component Clq) (Agrawal & Volanakis, 1994), modified self (apoptotic cells) (Gershov et al, 2000; Bijl et al, 2003) and non-self cells (bacteria and viruses) (Hind et al, 1984). However, the role of CRP and SAP remains poorly understood.
the PTX3 molecule, also called TSG-14 (TNF-stimulated gene 14) has been identified as a molecule whose synthesis is greatly enhanced in fibroblasts and endothelial cells in response to stimulation by TNFα or IL-1β (Breviario et al, 1992; Lee et al, 1993). PTX3 is the prototype for long pentraxins: the C-terminal area of PTX3 is homologous (17% identity) to whole CRP and contains a complementary N-terminal domain which is not homologous with any other molecule (Garlanda et al, 2005).
   b. The PTX3 Molecule

The PTX3 molecule is produced by many cell types such as endothelial cells, fibroblasts, chondrocytes, myocytes, mononuclear phagocytes, dendritic cells and epithelial cells in response to a pro-inflammatory stimulus or to a microbial constituent. (Breviario et al, 1992; Abderrahim-Ferkoune et al, 2003; Alles et al, 1994; Doni et al, 2003; Goodman at al, 2000; Vouret-Craviari et al, 1997; Nauta et al, 2005; Garlanda et al, 2005). The PTX3 molecule is produced in the form of a multimer consisting of 10 to 20 sub-units (Bottazzi et al, 1997).

Contrary to CRP and SAP, many functions have been described for the PTX3 molecule:
PTX3 binds to the Clq component of complement. This binding can either activate or inhibit complement classical pathway depending on the soluble or immobilised structure of Clq (Nauta et al, 2003),
PTX3 binds to apoptotic cells and favours their elimination by phagocytic cells (Rovere et al, 2000),
PTX3 binds to certain pathogens such as Salmonella typhimurium, Pseudomonas aeruginosa and Aspergillus fumigatus, thus facilitating their ingestion by phagocytic cells (Garlanda et al, 2002; Diniz et al, 2004).

Transgenic mice which overexpress PTX3 show increased survival in an endotoxemic model (Dias at al, 2001). In contrast, PTX3-deficient mice show increased sensitivity to pulmonary aspergillosis (Garlanda at al, 2002). This set of data shows that the PTX3 molecule acts like a soluble PRR and plays a crucial role in the recognition of modified self and non-self structures.

c. Anti-PTX3 Autoantibodies and Antineutrophil Cytoplasmic Antibodies(ANCA)-Associated Vasculitis (AAV)

ANCA-associated vasculitis (AAV) is a disorder characterized by necrotizing inflammation of small to medium-sized vessels. Examples of AAV include, without limitation, Wegener's granulomatosis (WGN), Churg-Strauss syndrome (CSS), and microscopic polyangiitis (MPA).

Patients affected by such disorder can be identified by using the criteria established by the Chapell Hill Conference (Jennette et al., 1994). Patients affected by Wegener's granulomatosis (WGN) typically present the following clinical symptoms:
granulomatous inflammation involving the respiratory tract,
and necrotizing vasculitis affecting small to medium-sized vessels (e.g. capillaries, venules, arterioles, and arteries).
   Necrotizing glomerulonephritis is common in such patients.

Patients affected by Churg-Strauss syndrome (CSS) typically present the following clinical symptoms:
eosinophil-rich and granulomatous inflammation involving the respiratory tract,
necrotizing vasculitis affecting small to medium-sized vessels as defined above,
asthma,
and eosinophilia.

Patients affected by microscopic polyangiitis (MPA) typically present the following clinical symptoms:
necrotizing vasculitis, with few or no immune deposits, affecting small vessels (i.e. capillaries, venules, arterioles),
and potentially necrotizing arteritis involving small and medium-sized arteries.
   Necrotizing glomerulonephritis is common in such patients, and pulmonary capillaritis often occurs.

ANCA-associated vasculitides (AAV) can usually be associated with serum positivity for anti-neutrophil cytoplasmic antibodies (ANCA) in patients presenting clinical symptoms of such diseases. In most cases, ANCA are directed against two constituents of neutrophil primary granules and monocyte lysosomes: myeloperoxydase (MPO) or proteinase 3 (PR3). Such ANCA have been identified both in the serum and plasma of patients affected by WGN (Lee et al., 2006). Although the pathophysiology of ANCA-associated vasculitides (AAV) remains elusive, neutrophils appear as targets and effectors of the auto-immune process. Diagnosis of MPA, WGN, and CSS can be based on pathological identification of pauci-immune small-vessels vasculitis. However, detection of anti-MPO and anti-PR3 autoantibodies is of major interest in the assessment of the disease. Using indirect immunofluorescence (IIF) on fixed neutrophils and ELISA, ANCA can be detected in most of the patients affected by MPA and WGN, but only in few patients affected by CSS (Kallenberg et al., 2006). In the absence of detectable ANCA, the diagnosis of “seronegative” AAV is thus difficult to assess. While some authors have reported that anti-MPO and -PR3 autoantibodies titers may correlate with disease activity, others have demonstrated that anti-MPO and -PR3 autoantibodies titers are not valuable to guide treatment. A reliable diagnosis of AAV is thus of high importance as the treatment of AAV involves immunosuppressive therapy responsible for severe adverse effects. Therefore, it seems crucial to identify new reliable biomarkers to establish a clear diagnosis of AAV, particularly in the case of seronegative AAV.

The inventors are the first to establish a method allowing the diagnosis of AAV, by quantifying the level of anti-PTX3 autoantibodies of subjects presenting clinical symptoms of such disease. This method is particularly useful for diagnosing seronegative AAV patients: indeed about half of AAV patients negative for anti-MPO and anti-PR3 autoantibodies present anti-PTX3 autoantibodies.

Furthermore, the inventors are the first to demonstrate that AAV patients present a specific pattern of ANCA, which differs from the classical perinuclear, atypical, and cytoplasmic ANCA (also known as p-, a-, and c-ANCA). This pattern is named “small-cytoplasmic” ANCA (scANCA). Said scANCA are typically characterized by a staining of small cytoplasmic granules when testing by indirect immunofluorescence (IIF) the serum of a subject presenting symptoms of AAV. These scANCA differ from the classical p- and a-ANCA as there is no homogenous perinuclear staining when performing such test, and differ as well from c-ANCA by the size of the stained cytoplasmic granules.

### II. DESCRIPTION OF THE INVENTION

A first aspect of the present invention relates to a method for in vitro diagnosis of an autoimmune response in a subject by detection in a biological fluid of said subject of autoantibodies directed against the pentraxin 3 antigen (PTX3) characterised in that the presence of antibodies directed against the PTX3 antigen (anti-PTX3 antibodies) is determined in biological fluids of said subject and the existence of an autoimmune response in the subject is concluded on this basis.

By autoimmune response, it is meant the existence of B lymphocytes producing antibodies directed against self-molecules.

By “anti-PTX3 antibodies”, it is meant according to the present invention any molecule containing a “paratope” capable of binding specifically to the PTX3 protein. By “anti-PTX3 antibodies”, it is also meant, according to the present invention, a homogeneous population of molecules which all contain the same “paratope” capable of binding specifically to the PTX3 protein.

The term “paratope” means the antigenic combination site contained in the Fab fragment of an antibody which is located in hypervariable or CDR regions of the V_{H} and V_{L} variable domains of an immunoglobulin heavy chain and light chain.

According to the present invention, the quantity of antibodies directed against the PTX3 antigen is determined in a biological fluid of the subject and the existence of an autoimmune response in the subject is established through comparison with the quantity of antibodies directed against the reference PTX3 antigen in the serum of healthy subjects.

Preferably, according to the present invention, the presence and/or quantity of antibodies directed against PTX3 is determined by detection of binding between the PTX3 antigen and the anti-PTX3 antibody.

The antigen-antibody binding reaction results from the interaction between antigen epitopes and antibody paratopes. This involves four types of non-covalent bonds (hydrogen bonds, electrostatic bonds, hydrophobic bonds and Van der Waals forces).

According to the present invention, detection of binding between the PTX3 antigen and the anti-PTX3 antibody is preferably carried out by immobilisation of the PTX3 antigen on a solid support, by precipitation reactions in liquid media and/or immunoprecipitation and/or by a gel precipitation reaction.

By precipitation reaction in liquid media, it is meant according to the present invention a reaction which consists in distributing equal quantities of a PTX3 antigen solution with increasing dilutions of a biological fluid, preferably an immune serum.

The equivalence zone (which is the point where the graph reaches its maximum) corresponds to the formation of an antigen-antibody binding.

The binding of a PTX3 antigen according to the invention to a solid support can be carried out using techniques well known to the person skilled in the art. The support can be in various forms, including bands or particles such as beads. The support surface can be polyfunctional or able to be polyfunctionalised in such a way as to bind the PTX3 antigen via covalent or non-covalent interactions which can be specific or non-specific.

For the purpose of illustration, the support on which the PTX3 antigen is immobilised can be a porous or non-porous material which is insoluble in water. The support can be hydrophilic or able to be made hydrophilic and can include inorganic powders such as silica, magnesium sulphate and aluminium; natural polymer materials, particularly cellulose and materials derived from cellulose; natural or synthetic polymers such as nitrocellulose, cellulose acetate, polyvinylchloride, polyacrylamide, reticulated dextran, agarose, polyacrylate, polyethylene, polypropylene, poly(4-methylbutene), polystyrene, polymethacrylate, polyethylene terephtalate, nylon, polyvinylbutyrite, certain types of glass such as Bioglass or ceramics.

The solid support for immobilisation of the PTX3 antigen is preferably of plastic or polypropylene type for ELISA or RIA assays, of membrane type for Western blot or Dot blot assays, of beads type or of foam type.

The term ELISA (Enzyme Linked ImmunoSorbent Assay) according to the present invention means an immunological test aimed at detecting and/or assaying anti-PTX3 antibodies in a biological fluid by immunoenzyme labelling.

The term RIA (Radioimmunology assay) according to the present invention means a radio-immunological test aimed at detecting and/or assaying anti-PTX3 antibodies in a biological fluid, based on the same principle as ELISA but using radio-immunology labelling.

The term fluorimetric assay according to the present invention designates the counting of particles, for example beads, which carry the PTX3 antigen on their surface and to which anti-PTX3 antibodies that may be present in a sample to be tested will have bound. The anti-PTX3 antibodies are detected by means of human anti-immunoglobulin antibodies coupled to a fluorochrome. Measurement of the quantity of anti-PTX3 antibodies is based on the intensity of fluorescence. This is carried out using a fluorescence analyser or cell analyser: flow cytometer or FACS (fluorescence activated cell sorter).

The term Western blot or immunotransfer according to the present invention means a technique by means of which the PTX3 antigen is separated by polyacrylamide gel electrophoresis then electrophoretically transferred onto a membrane (nitrocellulose for example). The deposition on the membrane of the biological fluid sample to be tested then makes it possible to bind anti-PTX3 antibodies that might be present in the sample to the PTX3 antigen. This is followed by detection by a second antibody labelled by an isotope, a fluorochrome or an enzyme.

The term Dot blot according to the present invention means an absorption technique which consists in depositing proteins on a nitrocellulose membrane in the form of dots then carrying out the usual immunoblot steps.

In addition, the presence and/or quantity of anti-PTX3 antibodies is determined according to the present invention by using a chromogenic substrate, by chemoluminescence, by fluorescence or by radio-labelling.

The term chromogenic substrate means the chromogenic substrate of an enzyme which eventually results in a coloured reaction detected by means of a spectrophotometer. For example, this can include X-gal/OPTG and salmon-gluc/O-Me-b-Gluc, OPD, ABTS.

The term chemoluminescence means a chemical reaction accompanied by light emission. Measurement of the light emitted makes it possible to quantify one of the reagents if the quantity of the other reagent is known. For example, this can include oxido-reduction of luminal (3-aminophthalhydrazide) with oxygenated water, for example, or any hydroxide.

The term fluorescence means the use of any fluorescent molecule such as the molecules described by ICHINOSE et al. (1991) or fluorescent derivatives of isothiocyanate, phycoerithrine, rhodamine isothiocyanate, dansyle chloride or compound XRITC, protein GFP (Green Fluorescent Protein) from the fish Aequorea Victoria and its many derivatives, or even protein YFP (Yellow Fluorescent Protein) as well as the protein luciferase.

The term radio-labelling refers to labelling with a radioactive substance. The radioactive substance can be labelled for example with an isotope chosen from among [³H], [³²P] and [¹²⁵I].

According to the present invention, ELISA or RIA assays make it possible to quantify the amount of anti-PTX3 antibodies and the Dot blot or Western blot type assays make it possible to detect higher quantities of anti-PTX3 antibodies compared to reference anti-PTX3 antibodies.

By “reference anti-PTX3 antibody quantity”, it is meant according to the present invention the quantity of anti-PTX3 antibodies obtained from the serum of a healthy subject, from a set of sera from healthy subjects or defined in an arbitrary manner.

By “healthy subject”, it is meant according to the present invention a subject without an autoimmune response.

The expression “anti-PTX3 antibody quantity defined in an arbitrary manner” according to the present invention means any method which allows reproduction of the mean value obtained with a pool of healthy subjects.

In an even more preferred manner, the gel precipitation reaction is chosen from among radial immunodiffusion, Ouchterlony double immunodiffusion, immunoelectrophoresis and fused rocket electrophoresis type reactions.

Bym radial immunodiffusion, also called the Mancini technique, is meant according to the present invention a gel precipitation reaction which consists in incorporating a PTX3 antigen solution in gelose and depositing a biological fluid likely to contain anti-PTX3 antibodies in wells. At equilibrium, a precipitation ring forms whose square diameter is proportional to the concentration of anti-PTX3 antibodies. Concentration is expressed by reference to a standard curve with anti-PTX3 antibodies of known concentration.

Ouchterlony double immunodiffusion according to the present invention means a gel precipitation reaction carried out as follows:

PTX3 antigen and anti-PTX3 antibody solutions are deposited in wells at a distance from each other in agarose gel. Molecules diffuse into the gel as a function of their size and form precipitation lines for each antigen and antibody system.

Each precipitation line corresponds to the respective equivalence zone, in other words to formation of an antigen-antibody binding.

This method makes it possible to carry out analysis of a biological fluid and identification of anti-PTX3 antibodies.

According to the present invention, immunoelectrophoresis means a gel precipitation reaction which involves separation of proteins by electrophoresis in agarose gel, followed by double diffusion against specific antibodies in a direction that is perpendicular to the axis of electrophoretic migration. Each equivalence zone corresponds to an antigen-antibody precipitate which results in a precipitation arc. Immunoelectrophoresis makes it possible to characterise or identify anti-PTX3 antibodies but is not a quantitative method.

According to the present invention, fused rocket electrophoresis means a gel precipitation reaction in which the PTX3 antigen incorporated into agarose gel is immobile (as a result of gel pH) and the negatively charged anti-PTX3 antibody migrates under the effect of an electric field.

The resulting precipitation arc is in a fused rocket form whose height is proportional to the concentration of anti-PTX3 antibodies.

According to the present invention, biological fluid means blood, serum, plasma, lymph, urine, saliva, cerebrospinal fluid, preferably serum or plasma, and even more preferably serum.

In a preferred embodiment of the invention, the diagnostic method according to the invention is an ELISA assay and comprises the following steps:

a) Incubating serum with PTX3 antigens fixed on a solid support,

b) Washing the serum antibodies not fixed to PTX3 antigens of the solid support.

c) Adding anti-immunoglobulin antibodies coupled to a marker, said anti-immunoglobulin antibodies being capable of recognising serum antibodies.

d) Washing the anti-immunoglobulin antibodies not fixed to the solid support.

e) Detecting and/or quantifying the marker bound to the solid support and correlating it to the presence and/or quantity of serum antibodies.

The concentration is variable and is to be determined as a function of the support and PTX3 source.

The PTX3 antigens according to the present invention are chosen from whole PTX3 of human, animal or synthetic origin, one or more PTX3 fragments of human, animal or synthetic origin, and PTX3 homologue molecules, preferentially chosen from the pentraxins family and/or presenting substantial homology with the primary, secondary or tertiary sequences. Examples of PTX3 fragment according to the present invention are the N-terminal domain of PTX3, such as the fragment of amino-acid sequence SEQ ID NO:1 (SEQ ID NO:1: MHLLAILFCALWSAVLAENSDDYDLMYVNLDNEIDNGLHPTEDPTPCDCGQEHSEWDKLF IMLENSQMRERMLLQATDDVLRGELQRLREELGRLAESLARPCAPGAPAEARLTSALDEL LQATRDAGRRLARMEGAEAQR), and the whole human PTX3 protein of sequence SEQ ID NO:2 (SEQ ID NO:2: MHLLAILFCALWSAVLAENSDDYDLMYVNLDNEIDNGLHPTEDPTPCDCGQEHSEWDKLF IMLENSQMRERMLLQATDDVLRGELQRLREELGRLAESLARPCAPGAPAEARLTSALDEL LQATRDAGRRLARMEGAEAQRPEEAGRALAAVLEELRQTRADLHAVQGWAARSWLPAGCE TAILFPMRSKKIFGSVHPVRPMRLESFSACIWVKATDVLNKTILFSYGTKRNPYEIQLYL SYQSIVFVVGGEENKLVAEAMVSLGRWTHLCGTWNSEEGLTSLWVNGELAATTVEMATGH IVPEGGILQIGQEKNGCCVGGGFDETLAFSGRLTGFNIWDSVLSNEEIRETGGAESCHIR GNIVGWGVTEIQPHGGAQYVS).

The PTX3 antigens according to the present invention are obtained from a prokaryotic recombinant system, for example in an Escherichia coli strain, a eukaryotic recombinant system, preferably CHO cells and line NSO, from purification using human or animal cells, tissues or biological fluids, or by chemical synthesis, preferably peptide synthesis.

According to the present invention, peptide synthesis means solid phase synthesis of PTX3 peptides.

The anti-immunoglobulin antibodies according to the present invention are selected from the group comprising anti-immunoglobulin G, anti-immunoglobulin A, anti-immunoglobulin M, anti-immunoglobulin D, and anti-immunoglobulin E.

In this preferred embodiment of the invention, the solid support consists of microbeads or microtitre plates, such as ELISA plates, and the marker is selected from the group comprising fluorescent, chemoluminescent, enzymatic and radioactive markers.

More particularly, in this preferred embodiment of the invention, the solid support is an ELISA plate and consists of the following steps:

a) Incubating the serum with PTX3 antigens fixed to the ELISA plate;

b) Washing the serum antibodies not fixed to PTX3 antigens of the ELISA plate;

c) Adding anti-immunoglobulin antibodies coupled to an enzyme, said anti-immunoglobulin antibodies being capable of recognising serum antibodies;

d) Washing the anti-immunoglobulin antibodies not fixed to the ELISA plate;

e) Adding the soluble substrate corresponding to the enzyme; and

f) Reading the absorbance values of wells of the ELISA plate in an ELISA reader at an appropriate wavelength and correlating it to the presence and/or quantity of antibodies in the serum.

Even more particularly, when the absorbance values for a subject presenting clinical symptoms of an antineutrophil cytoplasmic antibodies(ANCA)-associated vasculitis (AAV) is higher than the mean absorbance value for one or more healthy subject(s) plus 2 standard deviations, the existence of AAV in said subject is concluded.

In addition, corresponding enzymes and soluble substrates according to this preferred embodiment of the invention are selected from the group comprising:
Alkaline phosphatase and soluble substrate 4-NitroPhenyl Phosphate (PNPP)
Peroxidase and soluble substrate orthophenylene diamine (OPD)
β-galactosidase and soluble substrate 2-nitrophenyl β-galactoside (ONPG)
Glucose 6-phosphate dehydrogenase and soluble substrate glucose-6-phosphate (G6P).
Biotin and soluble substrate streptavidin coupled to peroxidase and substrate ABTS, OPD or TMB (3,3,5,5′-tetramethyl-benzidin) of peroxidase.

According to the present invention, the subject is preferably a mammal, still more preferably a human being.

According to the present invention, the anti-immunoglobulin antibodies are preferentially anti-human immunoglobulin antibodies.

According to the present invention, the diagnosis of an autoimmune response can be combined with the existence or prediction of an autoimmune disease in the subject. In a preferred manner, the autoimmune diseases are selected from the group comprising Gougerot-Sjögren syndrome, type 1 diabetes, monoclonal gammapathy, Wegener's granulomatosis (WGN), disseminated lupus erythematosus, atheromatous disease, Crohn's disease, Horton's disease, Reiter's disease (conjunctivo-uretro-synovial syndrome), rheumatoid arthritis, haemorrhagic recto-colitis, psoriatic rheumatism, sarcoidosis, sclerodermy, multiple sclerosis and autoimmune bullous dermatoses, Basedow's disease (hyperthyroidism), Hashimoto's chronic thyroiditis (hypothyroidism), Goodpasture's syndrome, pemphigus, myasthenia, insulin resistant diabetes, autoimmune haemolytic anaemia, autoimmune thrombocytopenic purpura, polymyositis and dermatomyositis, Biermer's anaemia, glomerulonephritis, certain sterile diseases, periarteritis nodosum and Churg-Strauss syndrome (CSS), microscopic polyangiitis (MPA), Still's disease, atrophying polychondritis, Behçet's disease and spondylarthritis.

According to a preferred embodiment, the diagnosis of an automimmune response is associated with the existence of an antineutrophil cytoplasmic antibodies (ANCA)-associated vasculitis in a subject presenting clinical symptoms of ANCA-associated vasculitis, said vasculitis being preferably selected from the group consisting of Wegener's granulomatosis (WGN), Churg-Strauss syndrome (CSS), and microscopic polyangiitis (MPA).

Said diagnosis can be carried out according to the method of the invention, i.e.:
the quantity of antibodies directed against pentraxin antigen (anti-PTX3 antibodies) is determined in a biological fluid of a subject presenting clinical symptoms of ANCA-associated vasculitis (AAV);
the quantity of anti-PTX3 antibodies of said subject is compared with a quantity of reference of anti-PTX3 antibodies as defined above; and
the existence of said antineutrophil cytoplasmic antibodies (ANCA)-associated vasculitis is concluded on this basis.

All the embodiments of the method of the invention as described above applies for the diagnosis of an automimmune response associated with the existence of an antineutrophil cytoplasmic antibodies (ANCA)-associated vasculitis in a subject presenting clinical symptoms of AAV.

According to a particular embodiment, the detection of antineutrophil cytoplasmic antibodies (ANCA) is researched in a biological fluid of said subject prior or after determining said quantity of anti-PTX3 antibodies. Preferably, said detection is carried out by indirect immunofluorescence (IIF). Even more preferably, the ANCA identified in said subject are small cytoplasmic ANCA (scANCA).

The method for diagnosis of an autoimmune response associated with the existence of an autoimmune disease, and more particularly of AAV, in a subject according to one aspect of the present invention can also include detection and/or quantification in the serum of said subject of antibodies directed against other autoantigens and preferentially the myeloperoxidase antigen (MPO) and/or proteinase 3 antigen (PR3) and/or elastase and/or BPI and/or cathepsin G and/or nuclear antigens and/or lactoferrin.

According to the present invention, diagnosis of an autoimmune response can also be associated with a pathology characterised by tissue damage in the subject. In this case, and in a preferred manner, tissue damage in the subject is due to necrosis, particularly infarction, chronic inflammation or chronic infection.

Tissue damage is assessed by measuring the concentration in various biological fluids of molecules released when cells are damaged (these molecules can be enzymes such as transaminases or inflammatory proteins)

A second aspect of the present invention relates to a diagnostic kit for detection and/or quantification in a biological fluid of antibodies directed against the PTX3 antigen, comprising:

a) a solid support wherein the PTX3 antigen is fixed to the solid support.

The characteristics of the kit (solid support, PTX3 antigen, etc) according to the present invention are as defined above for the diagnostic method.

According to the present invention, biological fluid means blood, serum, plasma, lymph, urine, saliva, cerebro-spinal fluid, preferentially serum.

Optionally, the kit according to the present invention comprises a solution containing one or more saturation proteins which saturate the reactive sites of the solid support.

Optionally, the kit according to the present invention further comprises:

b) a solution containing anti-immunoglobulin antibodies conjugated to a marker.

According to the present invention, the anti-immunoglobulin antibodies used in the kit are preferably selected from the group comprising anti-immunoglobulin G, anti-immunoglobulin A, anti-immunoglobulin M, anti-immunoglobulin D, and anti-immunoglobulin E.

Optionally, the kit according to the present invention further comprises:

c) a washing solution.

According to the present invention, a washing solution means a buffered saline solution containing a low concentration of a detergent and/or a saturation protein, preferentially of the bovine serum albumin or gelatin type.

A “saturation protein” means a protein which saturates the reactive site of the solid support.

Preferably, the solid support used in the kit according to the present invention is an ELISA plate and the marker is an enzyme.

Optionally, the kit according to the present invention further comprises:

d) a solution containing the soluble substrate corresponding to the enzyme.

According to a preferred embodiment, the kit is for the diagnosis of an autoimmune response associated with the existence of an ANCA-associated vasculitis (AAV) in a subject presenting clinical symptoms of said AAV.

A third aspect of the present invention relates to the use of a PTX3 antigen for diagnosis of an autoimmune disease in humans. Preferably, said autoimmune disease is an ANCA-associated vasculitis.

Preferably, an ELISA test is used for the diagnosis.

A fourth aspect of the present invention relates to the use of the diagnostic method or kit according to the present invention to identify, prior to the appearance of clinical symptoms, subjects at risk from an autoimmune response.

Preferably, the diagnostic method or kit according to the present invention is used to monitor the evolution of an autoimmune response, predict progression of the disease and/or monitor the efficacy of treatment.

The term “progression of the disease” means aggravation of clinical signs.

### BRIEF DESCRIPTION OF THE FIGURES

FIG. 1: Detection of anti-PTX3 antibodies in subjects presenting positive indirect immunofluorescence (IFI) associated with anti-MPO autoantibodies or anti-PR3 autoantibodies

FIG. 2: detection of anti-PTX3 antibodies in subjects with positive IFI in the absence of anti-MPO autoantibodies or anti-PR3 autoantibodies.

FIG. 3: Prevalence of anti-PTX3 autoantibodies in sera of AAV patients. Anti-PTX3 autoantibodies were detected by ELISA in 237 sera of AAV patients with MPA, WGN, or CSS and of 227 sera from healthy subjects (HS). Results are expressed in OD values. Dotted line corresponds to mean+2 SD titres of anti-PTX3 autoantibodies in HS sera; full line corresponds to the mean OD in each group of patients. ***p<10⁻⁴.

FIG. 4: Prevalence of anti-PTX3 autoantibodies in AAV patients. A, Anti-PTX3 autoantibodies were detected by ELISA in 150 AAV patients with MPA, WGN, or CSS and of 227 sera from healthy subjects (HS). When several samples were available for a single patient, the most positive one was considered in the figure. Results are expressed in OD values. Dotted line corresponds to mean+2 SD titres of anti-PTX3 autoantibodies in HS sera; full line corresponds to the mean OD in each group of patients. ***p<10⁻⁴. B, Prevalence of anti-PTX3 autoantibodies according to the disease. Results are expressed in percentage of patients with at least one positive serum for anti-PTX3 autoantibodies.

FIG. 5: Anti-pentraxin autoantibodies in AAV patients and specificity of anti-PTX3 autoantibodies. The presence of anti-CRP (A), -SAP (B) or -CRP and -SAP (C) autoantibodies were investigated by ELISA in 120 AAV sera (randomly selected among 237; anti-PTX3 autoantibodies were detected in 53 sera out of 120 [44.2%]) and in healthy subjects (HS). A&B, Dotted lines correspond to mean+2 SD titers of anti-CRP and -SAP autoantibodies in HS sera. Full lines correspond to the mean reactivity+SEM of sera in each group. ***p<0.001. C, Results are expressed in percentage of sera positive for anti-CRP or anti-SAP autoantibodies among anti-PTX3 positive (left column) and negative sera (right column). D, The titers of anti-PTX3 and —PTX3 N-terminal domain were compared in 24 AAV sera (randomly selected among 237).

FIG. 6: Immunofluorescence staining of neutrophils by anti-PTX3 Abs. Slides containing 4 biochips as substrate ethanol-fixed (upper left), formol-aceton fixed neutrophils (upper right) methanol-fixed neutrophils (lower left) and HEp2 cells (lower right) were incubated with A, a serum from a patient with anti-PTX3 autoantibodies and neither anti-MPO, -PR3 -BPI, -lactoferrine, -elastase nor -cathepsin G autoantibodies, B, an anti-PTX3 polyclonal antibody or C-E, control sera for p-ANCA (C), a-ANCA (D), c-ANCA (E). A, C-E, results are representative of the results obtained with one out of four sera. B, results are representative of one out of three experiments.

### EXAMPLES

### 1. Use of a Diagnostic Test Allowing Investigation of Anti-PTX3 Autoantibodies in Patient Sera

a. Description of the ELISA Test Allowing Investigation of Anti-PTX3 Antibodies

96-well ELISA plates (Maxisorb®; Nunc, Roskilde, Denmark) are incubated or not overnight at 4° C. with 100 μL of PTX3 at 10 μg/mL in carbonate/bicarbonate buffer pH=9.6. The wells are then emptied and incubated for 1 hour 30 min with 300 μL of a bovine serum albumin (BSA) solution at 1% in 10 mM phosphate saline buffer, pH=7.4. Sera from patients and healthy subjects are diluted to 1/400 in PBS buffer containing 0.5% BSA (w/v) and 0.05% Tween 20 (w/v). 100 μL of this dilution is deposited for each of the sera in a well coated with PTX3 and in an uncoated well (which allows determination of background noise for each serum sample). After incubation for 2 h at 37° C., the plates are washed 4 times with 200 μL of PBS containing 1% of Tween 20 (w/v) then incubated for 1 h30 at 37° C. with 100 μL of human anti-immunoglobulin antibody (G, A and M) coupled to biotin (Jackson ImmunoResearch, West Grove, Pa.). After 4 washings with 200 μL of PBS containing 1% of Tween 20 (w/v), 100 μL of a streptavidin solution coupled to peroxidase is incubated for 1 h at 37° C. (commercial solution diluted to 1/1000, BD Pharmingen, San Jose, Calif.). After 4 washings in PBS containing 1% Tween 20 (w/v), fixed antibodies are detected using freshly prepared ABTS substrate (Sigma, St Louis, Calif.). ELISA plates are read using a fluoreometer (λ=405 nm with a reference at λ=620 nm). The results are expressed in optical density units (OD).

An example of raw results obtained after reading is presented below. The reproducibility of results was verified by carrying out successive manipulations.

In general, results were analysed as follows:
For each ELISA plate, 20 to 30 serum samples from healthy subjects were tested in parallel to 20 to 30 samples from patients.
For each serum, the OD value obtained for the uncoated well (background noise BSA) was subtracted from the OD value obtained with wells coated with the PTX3 molecule in order to obtain a specific OD.
The specific OD values obtained with sera from healthy subjects were added together in order to establish the mean and standard deviation. A threshold value defined as the mean plus 2 standard deviations is calculated for each ELISA plate (mean+2 s.d.).
The specific OD values for patient sera are considered to be positive when the OD value is greater than the mean threshold value+2sd.

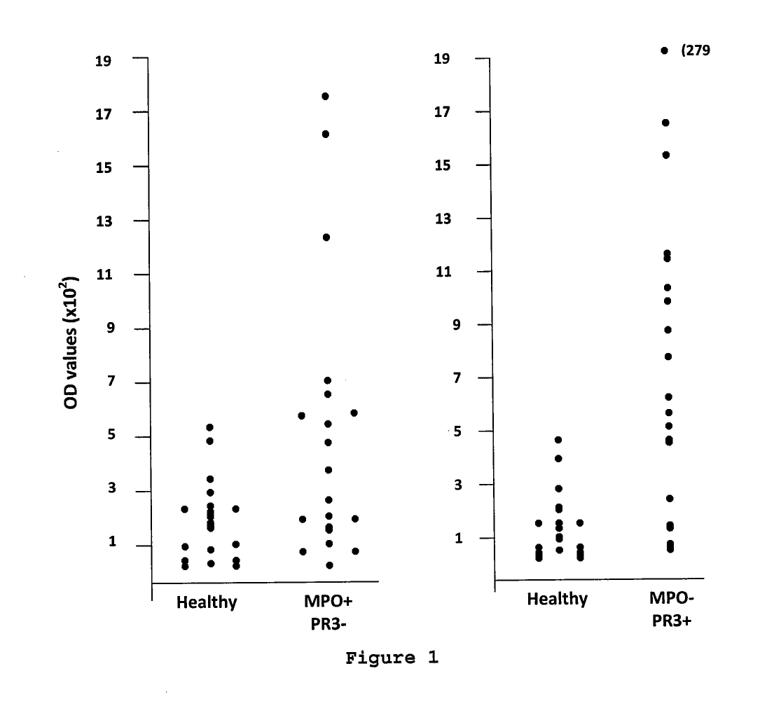

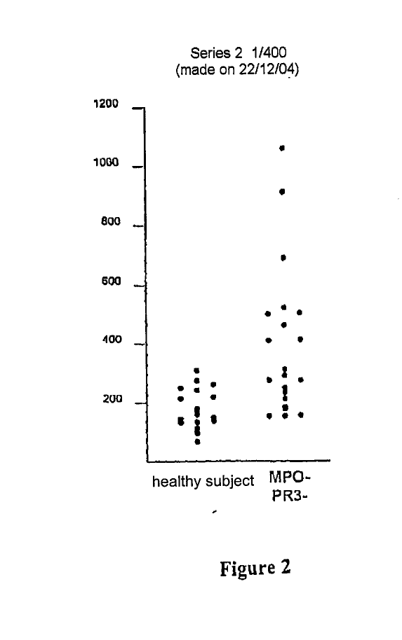

In the following examples, anti-PTX3 antibodies were investigated in patients presenting anti-neutrophil autoantibodies (IFI+, corresponding to ANCA+). 3 populations were analysed:
ANCA+, MPO+ (PR3−) patients, example a and FIG. 1, left-hand diagram.
ANCA+ PR3+ (MPO−) patients, example b and FIG. 1, right-hand diagram
ANCA+ PR3− MPG− patients, example c and FIG. 2.
example d: among the PR3− and MPO− patients, we re-analysed the preceding results (example c) as a function of the presence of autoantibodies directed against other specificities
presenting various autoantibodies.
   b. Presence of Anti-PTX3 Antibodies in IFI+ MPO+ (PR3−) Patients

Sera from 22 patients with anti-polynuclear autoantibodies (IFI+) and anti-MPO antibodies and sera from 23 healthy subjects were tested as described above. The threshold value (mean+2sd) is 0.037-9 patient sera were higher than the threshold value. The results obtained are presented in FIG. 1.

c. Presence of Anti-PTX3 Antibodies in IFI+ PR3+ (MPO−) Patients

Sera from 22 patients presenting with polynuclear autoantibodies (IF+) and anti-PR3 antibodies and from 23 healthy subjects were tested as described above. The threshold value (mean+2sd) is 0.04-11 patient sera were higher than the threshold value. The results obtained are presented in FIG. 1.

d. Presence of Anti-PTX3 Antibodies in IFI+ MPO− PR3− Patients

Sera from 21 patients presenting with polynuclear autoantibodies (IFI+) and 23 healthy subjects were tested as described above. The threshold value (mean+2sd) is 0.3-9 patient sera were higher than the threshold value. The results obtained are presented in FIG. 2.

e. Analysis of Anti-PTX3 Antibody Frequency in ANCA+ MPO− PR3− Patients

Investigation of autoantibodies directed against other specificities (bactericidal permeability increasing protein [BPI]), cathepsin G and elastase) was carried out in IFI+ MPO− and PR3− patients using a commercial ELISA kit (ANCAprofil; Euroimmun, Lübeck, Germany). In parallel, the presence of these anti-PTX3 antibody sera was investigated by ELISA, as described above.

The results obtained are represented in diagrammatic form below. They show that 72% of patients with IFI+ which cannot be confirmed at present by routine commercial kits (allowing detection of autoantibodies directed against the following antigens: MPO3, PR3, BPI, cathepsin G and elastase) have anti-PTX3 antibodies.

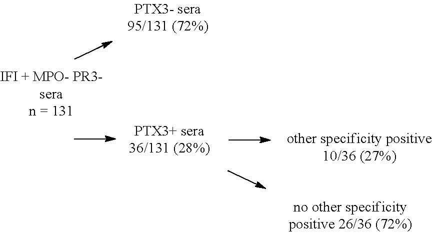

f. Anti-PTX3 in Patients Presenting with Autoantibodies Directed Against Various Autoantigens.

Investigation of anti-PTX3 autoantibodies was investigated by ELISA as described above in the sera of patients presenting various autoimmune diseases (and in particular systemic diseases). The sera tested were defined on the basis of the presence of:

anti-SSA autoantibodies: these autoantibodies are directed against the nuclear antigen SSA. They are found in Gougerot-Sjögren syndrome (dry syndrome), separately or associated with another connectivitis.

positive Farr test: which is indicative of the presence of native anti-DNA antibodies. These antibodies are present in 90% of disseminated lupus erythematosis (DLE) cases.

Cyclic citrulline antipeptide autoantibodies (anti-CCP): anti-CCP antibodies are a highly specific diagnostic tool for rheumatoid arthritis.

Anti-Saccharomyces cerevisiae antibodies (ASCA): ASCAs are directed against a structural epitope (Manα-1.2Man) ₙα-1-3Man, n= or >1 and are recognised as being associated with Crohn's disease.

The results obtained are summarised in Table 1.

TABLE 1
Serum autoantibodies (anti-SSA, anti-native DNA,
anti-Saccharomyces cerevisiae [ASCA], anti-CCP) are diluted
to 1/400 as described previously and tested in ELISA in
parallel to sera from healthy subjects.

| | | Number of | |
|---|---|---|---|
| | | sera | Frequency of |
| Serum | Number of | presenting | sera presenting |
| characteristics: | sera | anti-PTX3 | anti-PTX3 |
| presence of | tested: | antibodies | antibodies |
| Anti-SSA | 11 | 2 | 2/11 |
| Anti-native ADN | 10 | 4 | 4/10 |
| ASCA | 10 | 3 | 3/10 |
| Anti-CCP | 10 | 1 | 1/10 |

### 2. Use of a Diagnostic Test Allowing Investigation of Anti-PTX3 Autoantibodies in Patients Presenting Clinical Symptoms of Vasculitis.

a. Materials and Methods
i. Patient Sera

237 sera from 150 patients were obtained from the Immunology laboratories of the University Hospital of Angers (France), from the General Hospital of Le Mans (France) and from The National Referral Center for Necrotizing Vasculitis and Systemic Sclerosis (Cochin Hospital, Paris, France). Classification of AAV was based on the criteria defined by the Chapel Hill consensus conference (Jennette et al., 1994). 136 sera from 78 patients with microscopic polyangiitis (MPA) were analyzed, as well as 74 sera from 54 patients with Wegener's granulomatosis (WGN), and 23 sera from 22 patients with Churg-Strauss syndrome (CSS). Sera were aliquoted and kept frozen to avoid repeated thawing-freezing. 227 sera from healthy donors (Blood Collection Centre, Angers, France) were used as controls. Exhaustive clinical data were available for 60, 42 and 13 patients suffering from MPA, WGN, and CSS, respectively.

ii. Detection of Anti-Pentraxin Autoantibodies by ELISA

96-wells plates (Nunc, Roskilde, Denmark) were either uncoated or coated overnight at 4° C. with 50 μL of 10 μg/ml pentraxin in 50 nM carbonate-bicarbonate buffer (pH=9.6) (Sigma Aldrich, Saint-Louis, Mo.). Recombinant human PTX3 of sequence SEQ ID No. 2 was produced and purified as previously described (Bottazzi et al., 1997). CRP and SAP were purchased from Millipore (Billerica, Mass.) and Calbiochem (Merck, Darmstadt, Germany), respectively. The 1-141 N-terminal peptide of PTX3 of sequence SEQ ID No. 1 was chemically synthesized (Synprosis, Marseille, France) and used at 5 μg/ml. After saturation with 200 μL 1% bovine serum albumin (Euromedex, Souffelweyersheim, France) in endotoxin-free PBS (Lonza, Verviers, Belgium), 100 μL of 1:400 PBS-diluted serum, 0.5% BSA, and 0.05% Tween 20 were added and incubated for 2 hours at 37° C. Plates were then washed 3 times with 200 μL PBS, 0.05% Tween 20, and incubated at 37° C. for 1 hour with biotin-labeled goat anti-human heavy chain immunoglobulin antibody (Jackson Immunoresearch, West Grove, Pa.) diluted in PBS, 0.5% BSA, and 0.05% Tween 20. After washing, streptavidin-HRP(R&D Systems, Minneapolis, Minn.) was added and incubated 1 hour at 37° C. Plates were washed and bound Abs were detected using the 3,3,5,5′-tetramethyl-benzidin (TMB) substrate. Optical density (OD) at λ=450 nm minus λ=570 nm was measured in a spectrophotometer. Result for each sample is expressed as the net OD value obtained after subtraction of the OD obtained with the antigen-free well to the OD obtained with pentraxin-coated well. Samples were considered positive when the OD value was higher than the mean OD value+2 standard deviations (SD) of healthy subject sera.

The sequence of 1-141 N-terminal peptide of PTX3 is as follows:

(SEQ ID NO: 1)
MHLLAILFCALWSAVLAENSDDYDLMYVNLDNEIDNGLHPTEDPTPCDC
GQEHSEWDKLFIMLENSQMRERMLLQATDDVLRGELQRLREELGRLAES
LARPCAPGAPAEARLTSALDELLQATRDAGRRLARMEGAEAQR

The sequence of recombinant human PTX3 is as follows:

(SEQ ID NO: 2)
MHLLAILFCALWSAVLAENSDDYDLMYVNLDNEIDNGLHPTEDPTPCDC
GQEHSEWDKLFIMLENSQMRERMLLQATDDVLRGELQRLREELGRLAES
LARPCAPGAPAEARLTSALDELLQATRDAGRRLARMEGAEAQRPEEAGR
ALAAVLEELRQTRADLHAVQGWAARSWLPAGCETAILFPMRSKKIFGSV
HPVRPMRLESFSACIWVKATDVLNKTILFSYGTKRNPYEIQLYLSYQSI
VFVVGGEENKLVAEAMVSLGRWTHLCGTWNSEEGLTSLWVNGELAATTV
EMATGHIVPEGGILQIGQEKNGCCVGGGFDETLAFSGRLTGFNIWDSVL
SNEEIRETGGAESCHIRGNIVGWGVTEIQPHGGAQYVS.

iii. ANCA Detection

All sera were tested for the presence of ANCA by ELISA (Anti-PR3 IgG ELISA, anti-MPG IgG ELISA, and ANCA-Profile ELISA IgG; EuroImmun, Lübeck, Germany). Briefly, 96 wells-plates coated with PR3, MPO, lactoferrin, elastase, BPI, or cathepsin G were incubated with 100 μl of each diluted sera for 30 minutes. After washing, 100 μl of HRP-labeled rabbit anti-human IgG was incubated for 30 minutes. Bound autoantibodies were detected with TMB and the OD values were determined as described above.

iv. Indirect Immunofluorescence

Wells containing ethanol-, formaldehyde- and methanol-fixed granulocytes and HEp-2 cells (EUROPLUS™ Granulocyte Mosaic 23; Euroimmun) were incubated with 2.5 μg/ml rabbit anti-human PTX3 polyclonal Abs in PBS containing 0.5% Tween 20, for 30 minutes, at room temperature. After washing, 20 μg/ml FITC-labeled goat anti-rabbit immunoglogulin antibody (Invitrogen, Carlsbad, Calif.) was incubated for 30 minutes at room temperature. After washing and inclusion in glycerol, fluorescence was analyzed by microscopy (Colibri; Carl Zeiss, Göttingen, Germany). The same procedure was used with diluted human sera using a DyeLight488-labeled anti-human immunoglobulin as secondary antibody (Euroimmun). Sera giving rise to cytoplasmic (c-ANCA), perinuclear (p-ANCA), and atypical (a-ANCA) fluorescence staining patterns (from the collection of the Immunology and Allergology Department, University Hospital of Angers, France) were used as controls.

v. Statistical Analysis

Results are expressed as mean OD value±SD or SEM. Statistical analysis were performed using Prism 5 software (GraphPad software Inc.). Mann Whitney test was used to compare variables between groups of healthy subjects and vasculitis patients and Kruskall-Wallis test was used to compare variables in more than two groups. Wilcoxon tests were performed to compare paired data and correlations were calculated with Pearson's rank correlation. Differences were considered significant when p<0.05.

b. Results
i. Global Detection of Anti-PTX3 Autoantibodies in Sera from Patients with AAV.

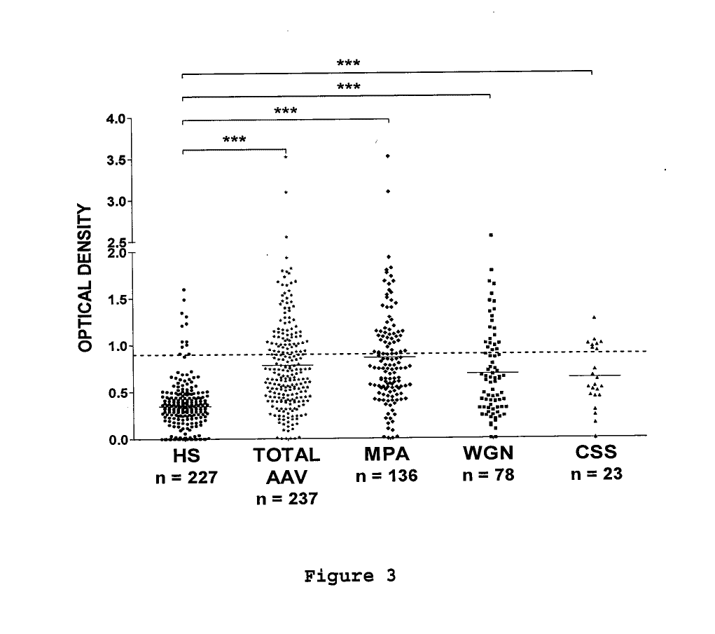

The presence of anti-PTX3 autoantibodies in 237 sera from AAV patients (Table 2) and 227 sera from healthy subjects was investigated by ELISA. The levels of anti-PTX3 autoantibodies were significantly higher in AAV patients than in healthy subjects (mean OD±SD=0.78±0.27, n=237 vs 0.34±0.51, n=227; p<10-4) (FIG. 3). Anti-PTX3 autoantibodies were detected in 82 of 237 (34.6%) sera from AAV patients and in 12 of 227 (5.3%) sera from healthy subjects. As MPA, WGN, and CSS differ by their prognosis and clinical features, we then analyzed the prevalence of anti-PTX3 autoantibodies in each group. Anti-PTX3 autoantibodies were detected in 38.2%, 33.3%, and 28.2% of sera from patients with MPA, WGN, and CSS, respectively (mean OD±SD=0.86±0.55, n=136; 0.68±0.48, n=78; and 0.64±0.33, n=23; respectively; p<10-4) (FIG. 3). We also detected anti-PTX3 autoantibodies in 33.7% of anti-MPO aAb positive sera and in 35.1% of anti-PR3 aAb positive sera (data not shown).

TABLE 2
Demographic and clinical features of AAV patients

| | CSS | WGN | MPA |
|---|---|---|---|
| Total | | | |
| Number of patients | 22 | 54 | 74 |
| Number of sera | 23 | 78 | 136 |
| Anti-MPO+ and/or anti-PR3+ | 13% | 71.8% | 66.2% |
| (ELISA) | | | |
| Patients with exhaustive clinical | | | |
| data | | | |
| Number of patients | 13 | 42 | 60 |
| Number of sera | 13 | 64 | 99 |
| Male/Female (number) | 9/4 | 20/22 | 32/28 |
| Mean age at diagnosis (years) | 47.3 | 52.7 | 60.9 |
| Active (mean BVAS) | 8 (13) | 36 (16) | 54 (16) |
| Non active disease (mean BVAS) | 5 (0) | 28 (0) | 45 (0) |
| Anti-MPO⁺ and/or anti-PR3⁺ | 23.1% | 76.2% | 70% |
| (ELISA) | | | |
| Clinical manifestations | % | % | % |
| General signs | 76.9 | 76.2 | 76.7 |
| Joints | 23.1 | 52.4 | 53.3 |
| Cutaneous | 61.5 | 45.2 | 41.7 |
| Mucous membranes/Eye | 0 | 30.9 | 10 |
| ENT | 69.2 | 69 | 13.3 |
| Chest | 84.6 | 64.3 | 41.7 |
| Cardio-vascular | 38.5 | 2.4 | 11.7 |
| Abdominal | 30.8 | 7.1 | 15 |
| Renal | 15.4 | 59.5 | 68.3 |
| Nervous system | 53.8 | 23.8 | 38.3 |

ii. Individual Detection of antiPTX3 Autoantibodies in AAV.

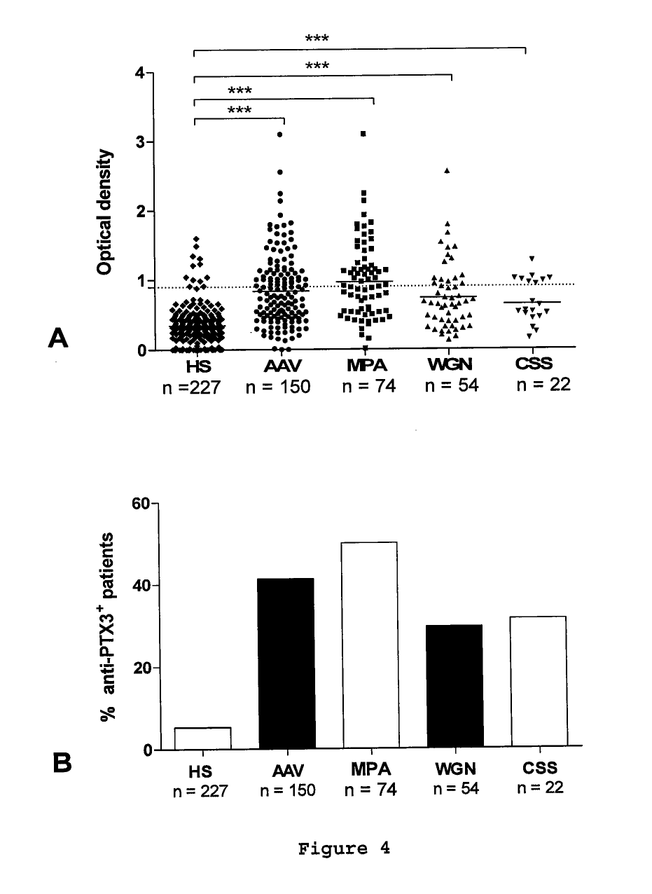

For some patients, 2 to 10 sera were collected at different time of the disease. Taken this information into account, anti-PTX3 autoantibodies were detected in 50.0%, 29.6%, and 31.8% of patients with MPA, WGN, and CSS, respectively (FIG. 4).

iii. Analysis of Anti-PTX3, Anti-CRP and Anti-SAP Autoantibodies in AAV.

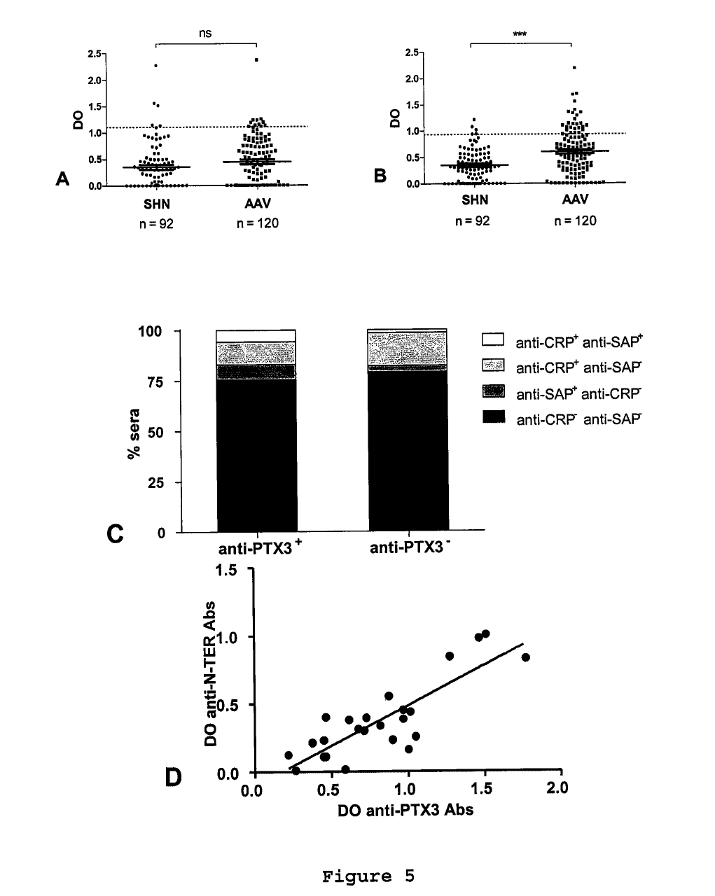

SAP and CRP belong to the pentraxin family. Anti-CRP and -SAP autoantibodies have been reported in patients with systemic lupus erythematosus. It was therefore evaluated whether circulating anti-CRP and/or -SAP autoantibodies were detectable in AAV. Anti-PTX3, -CRP, and -SAP autoantibodies were evaluated by ELISA in 120 randomly selected sera of AAV patients. Anti-CRP aAb titers were not significantly different in AAV patients and healthy subjects (mean OD±SD=0.44±0.45 vs 0.35±0.42, respectively) (FIG. 5A). Moreover, although anti-SAP aAb titers were higher in AAV patients than healthy subjects (mean OD±SD=0.60±0.43 vs 0.35±0.30; p<0.001), only 17.5% of the AAV sera were positive for anti-SAP autoantibodies (FIG. 5B) (while 44.2% were positive for anti-PTX3 autoantibodies; data not shown).

iv. Cross Reactivity of the Anti-PTX3 Autoantibodies.

The pentraxins PTX3, CRP, and SAP share 17% amino-acid homology in the C-terminal domain. It was thus needed to exclude that PTX3 immunoreactivity did not result from a recognition of PTX3 by anti-short pentraxin autoantibodies. First, among 53 anti-PTX3 aAb positive sera, 40 (75.5%) were negative for both anti-CRP and -SAP autoantibodies (FIG. 5C and Table 3). Second, titers of autoantibodies directed against the N-terminal domain of PTX3 (absent in CRP and SAP) correlate with anti-PTX3 aAb titers (r=0.75, p<0.001) (FIG. 5D). These results suggest that most of the anti-PTX3 autoantibodies detected bind selectively to PTX3.

TABLE 3
Presence of anti-CRP, -SAP, and -PTX3
autoantibodies in 120 sera of AAV patients.

| | | | | anti-CRP | anti-CRP |
|---|---|---|---|---|---|
| | | | | aAbs⁺ | aAbs⁻ |
| | | anti-CRP | anti-SAP | anti-SAP | anti-SAP |
| | | aAbs⁺ | aAbs⁺ | aAbs⁺ | aAbs⁻ |
| | | (%) | (%) | (%) | (%) |
| anti-PTX3 aAbs⁺ | 53 | 4 (7.5) | 6 (11.3) | 3 (5.7) | 40 (75.5) |
| anti-PTX3 aAbs⁻ | 67 | 2 (3.0) | 11 (16.4) | 1 (1.5) | 54 (79.1) |
| TOTAL | 120 | 6 (5.0) | 17 (14.2) | 4 (3.3) | 96 (77.5) |

v. Prevalence of Anti-PTX3 Autoantibodies in ANCA-Negative Vasculitis.

In patients with clinical symptoms of vasculitis, the presence of anti-MPO and/or -PR3 autoantibodies constitutes strong evidence in favor of the diagnosis of AAV. However, at the time of diagnosis these two specificity of ANCA are not detected in 5 to 15% of patients with confirmed MPA or WGN and in most of the patients with CSS. We thus investigated whether such ANCA negative patients may have anti-PTX3 autoantibodies. Among the 237 AAV sera, 15 sera from 15 patients (3 CSS, 10 MPA, and 2 WGN), taken at the time of diagnosis (before and during the first month of treatment), were negative for both anti-MPO and -PR3 autoantibodies and also for anti-lactoferrin, -BPI, -elastase, and -cathepsin G autoantibodies. Interestingly, anti-PTX3 autoantibodies were detected in 7 (5 MPA and 2 CSS) of these 15 sera.

vi. Fluorescence Pattern of Anti-PTX3 Autoantibodies on Fixed-Neutrophils.

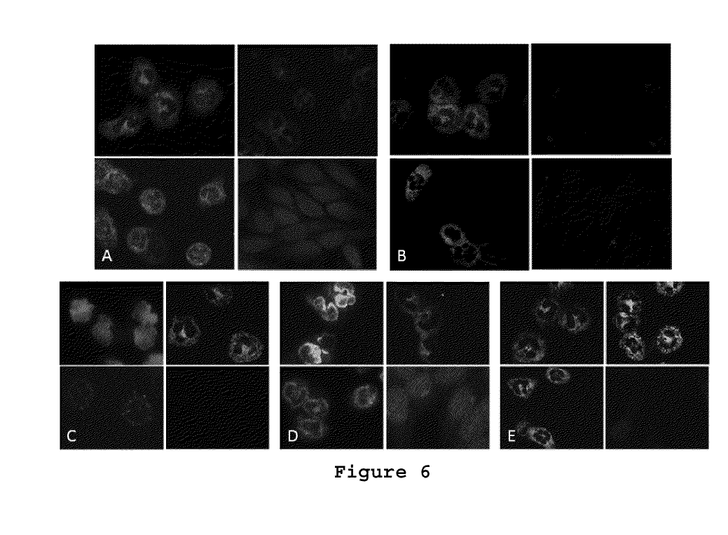

Indirect immunofluorescence (IIF) on human fixed-neutrophils remains the gold standard method for ANCA detection. It was therefore evaluated whether the presence of anti-PTX3 autoantibodies could be associated with a specific immunofluorescence staining pattern. Sera from 7 patients with anti-PTX3 autoantibodies and negative anti-MPO, -PR3, -BPI, -lactoferrin -cathepsin G and -elastase autoantibodies assessed by specific ELISA were incubated with human neutrophils fixed in methanol, ethanol or formol-acetone. A staining of small cytoplasmic granules was observed in methanol- and ethanol-fixed neutrophils in 4 out of 7 sera (FIG. 6A). The 3 other sera were IIF negative at the dilution tested (1/20). A similar fluorescent pattern was also observed with a rabbit anti-human PTX3 polyclonal Ab, used as a positive control (FIG. 6B). Fluorescence aspect obtained with the anti-PTX3 positive sera was different from classical p- and a-ANCA (FIGS. 6C and 6D) due to the absence of homogenous perinuclear staining on ethanol-fixed neutrophils and closer to c-ANCA (FIG. 6E). Nevertheless, the fluorescence aspect with anti-PTX3 positive sera differed from the c-ANCA pattern, as the cytoplasmic granules stained were smaller, visible in methanol and ethanol-fixed neutrophils, and less detectable in formol-fixed neutrophils (FIG. 6E). This typical staining was called sc-ANCA for small cytoplasmic ANCA.

c. Discussion

The results above demonstrate the presence of anti-PTX3 autoantibodies in patients presenting clinical symptoms of AAV, and that anti-PTX3 autoantibodies give rise to a specific pattern in IIF.

As PTX3, MPO, and PR3 colocalize in neutrophil granules, the presence of anti-PTX3 autoantibodies in AAV patients was investigated: anti-PTX3 autoantibodies were found in 50.0%, 29.6%, and 31.8% of the patients with MPA, WGN, or CSS, respectively.

Furthermore, it is known that 5 to 15% of the patients with pathological confirmation of WGN or MPA and 40% of patients with CSS are anti-MPO, and -PR3 autoantibodies negative. These results show that anti-PTX3 autoantibodies are present in nearly half of the sera of AAV patients without anti-MPO or anti-PR3 autoantibodies. These data thus highlight anti-PTX3 autoantibodies as a new biological marker, complementary to anti-MPO and -PR3 Abs, useful in the diagnosis of WGN, CSS, and MPA.

Additionally, PTX3 shares 17% amino acid homology with short pentraxins. It was thus important to exclude that autoantibodies against PTX3 could be mainly anti-CRP or anti-SAP autoantibodies cross-reacting with PTX3. The results show that anti-CRP and anti-SAP autoantibodies are not significantly detected in AAV.

The sera of AAV patients with anti-PTX3 autoantibodies show a IIF specific pattern visible in ethanol and methanol-fixed neutrophils and not in formol-fixed neutrophils, with smaller fluorescent granules than c-ANCA. The cytoplasmic pattern (c-ANCA) and the usual perinuclear pattern (p-ANCA) are commonly associated with Abs against PR3 and MPO, respectively. These preformed proteins colocalize in primary azurophilic granules of neutrophils. In contrast, PTX3 is stored in secondary, lactoferrin-positive granules. These different localisations may contribute to explain the specific pattern observed with anti-PTX3 Abs.

In conclusion, the presence of anti-PTX3 autoantibodies in AAV patients is demonstrated for the first time, especially in the sera of some ANCA-negative patients. Anti-PTX3 autoantibodies thus appear as a promising biomarker and may be used complementary to anti-MPO and -PR3 autoantibodies.

The Sequence Listings (SEQ ID NO:1 and SEQ ID NO:2) are submitted herewith as an ASCII text file (“358511D23604ProjetPatentInST25.txt”, created 15 Jun. 2011, and being 5,269 bytes), and herein incorporated by reference.

### BIBLIOGRAPHY

Abderrahim-Ferkoune A, Bezy O, Chiellini C, Maffei M, Grimaldi P, Bonino F, Moustaid-Moussa N, Pasqualini F, Mantovani A, Ailhaud G, Amri E Z. Characterization of the long pentraxin PTX3 as a TNFalpha-induced secreted protein of adipose cells. J Lipid Res. 2003, 44:994-1000.
Agrawal A, Volanakis J E. Probing the Clq-binding site on human C-reactive protein by site-directed mutagenesis. J. Immunol. 1994, 152:5404-10.
Alles V V, Bottazzi B, Peri G, Golay J, Introna M, Mantovani A. Inducible expression of PTX3, a new member of the pentraxin family, in human mononuclear phagocytes. Blood. 1994, 84:3483-93.
Bijl M, Horst G, Bijzet J, Bootsma H, Limburg P C, Kallenberg C G. Serum amyloid P component binds to late apoptotic cells and mediates their uptake by monocyte-derived macrophages. Arthritis. Rheum. 2003, 48:248-54.
Bottazzi B, Vouret-Craviari V, Bastone A, De Gioia L, Matteucci C, Peri G, Spreafico F, Pausa M, D'Ettorre C, Gianazza E, Tagliabue A, Salmona M, Tedesco F, Introna M, Mantovani A. Multimer formation and ligand recognition by the long pentraxin PTX3. Similarities and differences with the short pentraxins C-reactive protein and serum amyloid P component. J. Biol. Chem. 1997, 272:32817-23.
Breviario F, dAniello E M, Golay J, Peri G, Bottazzi B, Bairoch A, Saccone S, Marzella R, Predazzi V, Rocchi M, et al. Interleukin-l-inducible genes in endothelial cells. Cloning of a new gene related to C-reactive protein and serum amyloid P component. J Biol. Chem. 1992, 267:22190-7.
Dias A A, Goodman A R, Dos Santos J L, Gomes R N, Altmeyer A, Bozza P T, Horta M F, Vilcek J, Reis L F. TSG-14 transgenic mice have improved survival to endotoxemia and to CLP-induced sepsis. J. Leuk. Biol. 2001, 69:928-36.
Diniz S N, Nomizo R, Cisalpino P S, Teixeira M M, Brown G D, Mantovani A, Gordon S, Reis L F, Dias A A. PTX3 fonction as an opsonin for the dectm-1-dependent internalization of zymosan by macrophages. J Leukoc Biol. 2004 75, 649-56.
Doni A, Peri G, Chieppa M, Allavena P, Pasqualini F, Vago L, Romani L, Garlanda C, Mantovani A. Production of the soluble pattern recognition receptor PTX3 by myeloid, but not plasmacytoid, dendritic cells. Eux J. Immunol. 2003, 33:2886-93.
Garlanda C, Bottazzi B, Bastone A, Mantovani A. Pentraxins at the crossroads Between Imiate Immunity, Inflammation, Matrix Deposition and Female Fertility. Annu Rev Immunol. 2005, 23: 337-366.
Garlanda C, Hirscli E, Bozza S, Salustri A, De Acetis M, Nota R, Maccagno A, Riva F, Bottazzi B, Peri G, Doni A, Vago L, Botto M, De Santis R, Carminati P, Siracusa G, Altruda F, Vecchi A, Romani L, Mantovani A. Non-redundant role of the long pentraxin PTX3 in anti-fungal innate immune response. Nature. 2002, 420:182-6.
Gershov D, Kim S, Brot N, Elkon K B. C-Reactive protein binds to apoptotic cells, protects the cells from assembly of the terminal complement components, and sustains an antiinflammatory innate immune response: implications for systemic autoimmunity. J. Exp. Med. 2000, 192:1353-64.
Goodman A R, Levy D E, Reis L F, Vilcek J. Differential regulation of TSG-14 expression in murine fibroblasts and peritoneal macrophages. J Leukoc Biol. 2000, 67:387-95,
Hind C R, Collins P M, Renn D, Cook R B, Caspi D, Marilyn L Baltz, and Pepys M B, Binding specificity of serum amyloid P component for the pyruvate acetal of galactose. J Exp Med. 1984 Apr. 1; 159(4):1058-69.
Janeway C A Jr, Medzhitov R., Innate immune recognition. Annu Rev Immunol. 2002; 20:197-216. Epub 2001 Oct. 4. Review.
Lee G W, Lee T H, Vilcek J. TSG-14, a tumor necrosis factor- and IL-1-inducible protein. is a novel member of the pentaxin family of acute phase proteins. J. Immunol. 1993; 150:1804-12.
Matsushita M, Fujita T. The role of ficolins in innate immunity. Immunobiology. 2002, 205:490-7. Nauta A J, Bottazzi B, Mantovani A, Salvatori G, Kishore U, Schwaeble W J, Gingras A R, Tzirna S, Vivanco F, Egido J, Tijsma O, Hack E C, Daha M R, Roos A. Biochemical and functional characterization of the interaction between pentraxin 3 and Clq. Eur J. Immunol. 2003, 33:465-73.
Medzhitov R, Janeway C Jr., Innate immunity, N Engl J. Med. 2000 Aug. 3; 343(5):338-44. Review.
Nauta A J, de Haij S, Bottazzi B, Mantovani A, Bornas M C, Aten J, Rastaldi M P, Daha M R, van Kooten C, Roos A. Human renal epithelial cells produce the long pentraxin PTX3. Kidney Int. 2005, 67:543-53.
Rovere P, Peri G, Fazzini F, Bottazzi B, Doni A, Bondanza A, Zimmermann V S, Garlanda C, Fascio U, Sabbadini M G, Rugarli C, Mantovani A, Manfredi A A. The long pentraxin PTX3 binds to apoptotic cells and regulates their clearance by antigen-presenting dendritic cells. Blood. 2000, 96:4300-6.
Van de Wetering J K, van Golde L M, Batenburg J J. Collectins: players of the innate immune system. Eux. J. Biochem. 2004, 271:1229-49.
Vouret-Craviari V, Matteucci C, Peri G, Poli G, Introna M, Mantovani A. Expression of a long pentraxin, PTX3, by monocytes exposed to the mycobacterial cell wall component lipoarabinomannan. Infect Immun. 1997, 65:1345-50.
Jennette J C, Falk R J, Andrassy K, Bacon P A, Churg J, Gross W L, et al. Nomenclature of systemic vasculitides. Proposal of an international consensus conference. Arthritis Rheum 1994; 37(2):187-92.
Lee A S, Finkielman J D, Peikert T, Hummel A M, Viss M A, Jacob G L, Homburger H A, Specks U; Wegener's Granulomatosis Etanercept Trial Research Group. Agreement of anti-neutrophil cytoplasmic antibody measurements obtained from serum and plasma. Clin Exp Immunol. 2006 October; 146(1):15-20.
Kallenberg C G, Heering a P, Stegeman C A. Mechanisms of Disease: pathogenesis and treatment of ANCA-associated vasculitides. Nat Clin Pract Rheumatol 2006; 2(12):661-70.

1. A method for in vitro diagnosis of an autoimmune response associated with the existence of an antineutrophil cytoplasmic antibodies(ANCA)-associated vasculitis in a subject presenting clinical symptoms of said vasculitis, wherein:
the quantity of antibodies directed against the pentraxin 3 antigen(anti-PTX3 antibodies) is determined in a biological fluid of said subject;
said quantity of anti-PTX3 antibodies of said subject is compared with a quantity of reference of anti-PTX3 antibodies; and
the existence of antineutrophil cytoplasmic antibodies(ANCA)-associated vasculitis in said subject is concluded on this basis.

2. The method according to claim 1, wherein the quantity of antibodies directed against PTX3 is determined by detection of binding between the PTX3 antigen and the anti-PTX3 antibody.

3. The method according to claim 2, wherein detection of binding between the PTX3 antigen and the anti-PTX3 antibody is carried out by immobilisation of the PTX3 antigen on a solid support, by precipitation reactions in liquid media and/or immunoprecipitation and/or by a gel precipitation reaction.

4. The method according to claim 1, wherein for quantification of PTX3 antibodies, the method comprises an ELISA or RIA assay and in order to detect higher quantities of anti-PTX3 antibodies compared to the quantity of reference anti-PTX3 antibodies, the method comprises a Western blot or Dot blot type assay.

5. The method according to claim 1, wherein the quantity of reference anti-PTX3 antibodies is the quantity of anti-PTX3 antibodies obtained from the serum of a healthy subject, from a set of serum from healthy subjects, or defined in an arbitrary manner by any means which allows reproduction of a mean value obtained with a pool from healthy subjects.

6. The method according to claim 1, wherein the biological fluid is selected from the group consisting of blood, serum, plasma, lymph, urine, saliva, and cerebrospinal fluid.

7. The method according to claim 1, wherein the biological fluid is serum.

8. The method according to claim 1, wherein said method is an ELISA assay and comprises the following steps:
a) Incubating the subject's serum with PTX3 antigens fixed on a solid support,
b) Washing the serum antibodies not fixed to PTX3 antigens of the solid support,
c) Adding anti-immunoglobulin antibodies coupled to a marker, said anti-immunoglobulin antibodies being capable of recognising serum antibodies,
d) Washing the anti-immunoglobulin antibodies not fixed to the solid support,
e) Quantifying the marker bound to the solid support and correlating it to quantity of serum antibodies.

9. The method according to claim 1, wherein said PTX3 antigen is chosen from whole PTX3 of human, animal or synthetic origin, one or more PTX3 fragments of human, animal or synthetic origin, and PTX3 homologue molecules, preferentially from the pentraxins family and/or presenting substantial homology with the primary, secondary or tertiary sequences.

10. The method according to claim 9, wherein said PTX3 fragment is the N-terminal domain of PTX3.

11. The method according to claim 10, wherein said N-terminal domain of PTX3 is of sequence SEQ ID No. 1.

12. The method according to claim 8, wherein said marker is enzymatic and its corresponding soluble substrate are selected from the group consisting of:
Alkaline phosphatase and soluble substrate 4-NitroPhenyl Phosphate (PNPP)
Peroxidase and soluble substrate orthophenylene diamine (OPD)
β-galactosidase and soluble substrate 2-nitrophenyl β-galactoside (ONPG)
Glucose 6-phosphate dehydrogenase and soluble substrate glucose-6-phosphate (G6P).
Biotin and soluble substrate streptavidin coupled to peroxidase and substrate ARTS, OPD, or TME of peroxidase.

13. The method according to claim 1, wherein the subject is a mammal.

14. The method according to claim 1, wherein the subject is a human being.

15. The method according to claim 14, wherein the anti-immunoglobulin antibodies are anti-human immunoglobulin antibodies.

16. The method according to claim 1, wherein the antineutrophil cytoplasmic antibodies(ANCA)-associated vasculitis is selected from the group consisting of Wegener's granulomatosis (WGN), Churg-Strauss syndrome (CSS), and microscopic polyangiitis (MPA).

17. The method according to claim 1, wherein it also comprises the quantification in the serum of said subject of antibodies directed against the myeloperoxidase antigen (MPO) and/or proteinase 3 antigen (PR3) and/or elastase and/or bactericidal increasing protein (BPI) and/or cathepsin G and/or nuclear antigens, and/or lactoferrin.

18. The method according to claim 1, wherein the detection of antineutrophil cytoplasmic antibodies (ANCA) is researched in a biological fluid of said subject prior or after determining said quantity of anti-PTX3 antibodies.

19. The method according to claim 18, wherein said detection is carried out by indirect immunofluorescence (IFI);

20. The method according to claim 18, wherein said ANCA are small cytoplasmic ANCA (scANCA).


SEQUENCE LISTING (2 sequences)

SEQ ID NO: 1 - protein, 141 aa, Homo sapiens
  misc_feature: N-Terminal domain of human PTX3 protein
MHLLAILFCALWSAVLAENSDDYDLMYVNLDNEIDNGLHPTEDPTPCDCGQEHSEWDKLFIMLENSQMRERMLLQATDDVLRGELQRLREELGRLAESLARPCAPGAPAEARLTSALDELLQATRDAGRRLARMEGAEAQR

SEQ ID NO: 2 - protein, 381 aa, Homo sapiens
MHLLAILFCALWSAVLAENSDDYDLMYVNLDNEIDNGLHPTEDPTPCDCGQEHSEWDKLFIMLENSQMRERMLLQATDDVLRGELQRLREELGRLAESLARPCAPGAPAEARLTSALDELLQATRDAGRRLARMEGAEAQRPEEAGRALAAVLEELRQTRADLHAVQGWAARSWLPAGCETAILFPMRSKKIFGSVHPVRPMRLESFSACIWVKATDVLNKTILFSYGTKRNPYEIQLYLSYQSIVFVVGGEENKLVAEAMVSLGRWTHLCGTWNSEEGLTSLWVNGELAATTVEMATGHIVPEGGILQIGQEKNGCCVGGGFDETLAFSGRLTGFNIWDSVLSNEEIRETGGAESCHIRGNIVGWGVTEIQPHGGAQYVS